(19) United States
(12) Patent Application Publication
(10) Pub. No.: US 2026/0325990 A1
(43) Pub. Date: Oct. 22, 2026

(54) A STATIONARY PHASE FOR USE IN AFFINITY CHROMATOGRAPHY

(71) Applicant: Cytiva BioProcess R&D AB, Uppsala (SE)

(72) Inventors: Matthew David Daniel Miell, London Greater London (GB); Susana Brito Dos Santos, Stevenage Hertfordshire (GB); Peter Guterstam, Uppsala (SE)

(21) Appl. No.: 19/111,255

(22) PCT Filed: Sep. 27, 2023
(86) PCT No.: PCT/EP2023/076623

§ 371 Date: Mar. 12, 2025

(30) Foreign Application Priority Data
Sep. 28, 2022 (GB) 2214187.3

(51) Int. Cl. B01D 15/38; B01J 20/32

(52) U.S. Cl. CPC B01D 15/3809; B01J 20/3208; B01J 20/3274; B01J 20/3219

(57) ABSTRACT

A stationary phase for use in affinity chromatography for recovering adeno-associated virus (AAV) vectors (3) from a solution. The stationary phase comprises a porous polymer matrix (1) having a mean flow pore size of 0.1-2.0 μm, wherein the matrix (1) is functionalised through attachment of AAV vector ligands (2) to the matrix, the ligands (2) having a binding affinity for the AAV vector (3), and a density of ligands (2) in the polymer matrix (1) is 0.1 μmol/gram-mmol/gram.

### TECHNICAL FIELD

The present disclosure relates to a stationary phase for use in affinity chromatography for recovering adeno-associated virus (AAV) vectors from a solution, to a chromatograph comprising such stationary phase and to a process for recovering adeno-associated virus (AAV) vectors from a solution.

### BACKGROUND ART

In methods of gene therapy, delivery of a gene of interest into a cell requires the use of a vector, which may be derived from recombinant viruses such as adenovirus (AdV), adeno-associated virus (AAV) and lentivirus (LV). By means of the vector, a nucleic acid sequence (DNA or RNA) may be delivered to the cell where it undergoes processing by the biochemical machinery of the cell to alter its properties to yield the desired therapeutic effect.

The vector material is typically generated from cell lines that have been modified to produce the constituent parts of the vector e.g. its coat or capsid, and the nucleic acid material that is intended to be delivered to the cells.

After lysis of the host cells and clarification, the “crude materials” contains vectors, cell host debris, protein, genomic DNA, serum protein, some elements of medium, helper DNA, helper virus etc. These impurities may poison cells, reduce transduction efficiency, even induce systemic immune response or inflammatory response. Purity, efficacy and safety of clinical grade vectors is crucial. The vectors also need to maintain viral activity as intact as possible throughout the purification process. A variety of purification strategies have been developed, such as chromatographic separation methods.

In a porous bead-based chromatographic system, the binding event between a target entity and the solid phase/a ligand immobilised in the solid phase is dependent on diffusion into the porous bead, meaning binding capacity drops off with decreasing residence times. High flowrates are also particularly incompatible with porous beads at manufacturing scale where many litres of bead suspension are packed into a column.

Typical binding capacities for porous beads (using bovine serum albumin/monoclonal antibody, BSA/mAb) are in the region of 35-120 mg/ml dependant on the functionality of the solid phase and species bound. However, the low typical flowrates through such systems mean that overall productivities for single column porous bead systems of only around 10-120 mg/ml/min can be achieved.

A drawback of using porous beads is that the capacity of the material is dependent on the target's ability to access the inner surface area of the bead. Typical porous beads have pore sizes of between 15-30 nm and so have limitations in vector purification where the target vector can be much larger than the pore sizes.

Separations involving membranes and monoliths can be run at far higher flowrates than porous bead-based systems, typical residence times being in the order of 0.2-0.5 minutes. However, typical binding capacities at 10% breakthrough of target (mAb) for monoliths (10-20 mg/mL) and membranes (7.5-29 mg/mL) under dynamic flow are lower than porous beads. The inferior binding capacity of monolith and membrane materials (compared to porous bead-based materials) can be offset to some extent by utilising higher flowrates.

Additionally, although the pore sizes of membranes are far larger than porous beads, the binding capacity of the membranes decreases as the size of the adsorbed species increases. This emphasizes the need to have both highly porous adsorbents with a high accessible surface area.

There exists a need for chromatography materials that can efficiently purify these viral vectors, such as AAV vectors, to enable a therapeutic product to be recovered at industrial scale.

### SUMMARY OF THE INVENTION

It is an object of the present disclosure to provide a stationary phase for use in affinity chromatography for recovering adeno-associated virus (AAV) vectors from a solution, which stationary phase may be used to recover the AAV vector at industrial scale at sufficiently short residence times.

According to a first aspect there is provided a stationary phase for use in affinity chromatography for recovering adeno-associated virus (AAV) vectors from a solution, the stationary phase comprising a porous base matrix having a mean flow pore size of 0.1-2.0 μm, wherein the base matrix is functionalised through attachment of AAV vector ligands to the matrix, the ligand having a binding affinity for the AAV vector, and a density of ligands in the matrix is within the range from 0.1 μmol/gram to 10 mmol/gram.

The functionalised matrix is suitable for use as a stationary phase in affinity capture chromatography. In operation, the stationary phase comprising the functionalised matrix is contacted with a mobile phase, a solution, containing the AAV vector and the AAV vector is retained in the matrix by the AAV vector ligand in preference to other components also present in the solution. Such other components in the mobile phase may comprise impurities such as cell host debris, protein, genomic DNA, serum protein, some elements of medium, helper DNA, or helper virus, etc.

The stationary phase may be assembled in a capsule or cartridge that allows an even flow distribution over the stationary phase.

The above described stationary phase is a convective stationary phase, and the matrix may be a convection-based matrix, which includes any matrix in which application of a hydraulic pressure difference between the inflow and outflow of the matrix forces perfusion of the matrix, achieving substantially convective transport of the substance(s) into the matrix or out of the matrix. A convection-based matrix can be for example an adsorptive membrane where a flow through such materials is convective rather than diffusional. The present stationary phase has a high surface area for high binding capacity and a macroporosity needed for viruses to enter the matrix. When adding the mobile phase to the convective stationary phase, there is a convective flow of the mobile phase in the stationary phase, such that the mobile phase is directly in contact with the AAV vector ligands in the matrix. Thus, the AAV vectors in the mobile phase do not have to rely on diffusion to reach the AAV vector ligands.

Hence, the present invention enables chromatography materials for purification of AAV vectors which combine the high binding capacity traditionally associated with porous bead-based materials, with the higher flowrates that are achievable with monolith/membrane materials. The chromatography material can be made sufficiently porous so that the binding area is accessible to the large vectors, and suitably short residence times may be achieved.

The diameter of different viruses range from 20-300 nm. AAV vectors typically have a diameter of about 25 nm.

The base matrix may be a non-woven polymer matrix. Such a polymer matrix may be formed of polymer fibres, such as polymer nanofibers.

Mean flow pore (MFP) size is an indicator of material flow characteristics, and is measured by capillary flow porometry, based on the displacement of a wetting liquid with a known surface tension from the sample pores by applying a gas at increasing pressure. The higher the MFP size, the larger the flow of liquid through the material at a given pressure. The mean flow pore size is calculated from the point at which 50% of the flow goes through a sample. Mean flow pore size thus corresponds to the pore size calculated at the pressure where the wet curve and the half-dry curve meet.

In an alternative definition, the mean flow pore size of the present stationary phase may be seen as an effective pore size defined as the size of the largest sphere that is able to pass through the pore.

The mean flow pore size of the base matrix may be 0.1-1.8 μm, 0.1-1.6 μm, 0.1-1.4 μm, 0.1-1.2 μm, 0.1-1.0 μm, 0.1-0.8 μm, 0.1-0.6 μm, 0.1-0.4 μm, 0.1-0.2 μm, 0.2-2.0 μm, 0.4-2.0 μm, 0.6-2.0 μm, 0.8-2.0 μm, 1.0-2.0 μm, 1.2-2.0 μm, 1.4-2.0 μm, 1.6-2.0 μm, 1.8-2.0 μm, or 0.5-1.5 μm.

Advantageously, when adding the mobile phase (i.e., the solution comprising the AAV vector) to the stationary phase the rate of binding of the AAV vector to the AAV vector ligand is merely dependent on the binding kinetics. The matrix of the present stationary phase has an open pore structure where mass transfer is governed by convective flow. Hence, the use of the present stationary phase in affinity chromatography results in shorter residence times than when using traditional resin-based stationary phases. This results in cycle times of minutes instead of the hours needed for resin-based chromatography. Using the present stationary phase it may be possible to more than half the residence time as compared to when using resin-based stationary phases. Residence times as low as 1 second have been observed with the present stationary phase. This cuts weeks from lead times in process development.

The exact residence time may be dependent on which AAV serotype and mobile phase is used. The nanofiber polymer matrix may be selected from the following hydrophilic polymers: cellulose, polyethersulfone (PES), polystyrene, methyl acrylate, dextran and agarose.

The expression “AAV vector ligand”, or “AAV vector binding ligand” as used herein, refers to any molecule that has a suitable binding affinity for vectors based on one or more adeno-associated virus (AAV) based vectors, and which can be coupled to a chromatography material such as the present stationary phase. The AAV vector ligand may be a peptide or polypeptide, including an antibody or an antibody fragment, an oligonucleotide, such as DNA or RNA, such as an aptamer. The AAV vector ligand may be a camelid antibody or antibody fragment.

AAV vector binding ligands are known in the art. For example, POROS CaptureSelect AAVX resin (ThermoFischer Scientific) has demonstrated binding reactivity towards a set of AAV serotypes that includes AAV1 to AAV8, and AAVrh10. Other examples of resins incorporating affinity ligands include AVIPure® AAV2 affinity resin, AVIPure®AAV8 affinity resin and AVIPure® AAV9 affinity resin (Avitide/Repligen). As another example, Capto AVB and AVB Sepharose High Performance (Cytiva) are affinity resins with proven affinity for 10 adeno associated viruses from subclasses 1, 2, 3, and 5. The AVB ligand is a 14 kD fragment from a single chain camelid antibody.

The AAV vector ligand may be a polypeptide that is recombinantly produced, optionally in eukaryotic cells, e.g. in yest cells such as Saccharomyces cerevisiae.

The density of ligands in the functionalised polymer matrix may be from 0.1 μmol/gram to 1 mmol/gram, from 0.1 to 100 μmol/gram, from 0.1 to 10 μmol/gram, from 0.2 to 10 μmol/gram, from 0.5 to 5 μmol/gram, from 0.1 to 1 μmol/gram, from 0.5 to 2 μmol/gram, from 1 μmol/gram to 10 mmol/gram, from 10 μmol/gram to 10 mmol/gram, from 100 μmol/gram to 10 mmol/gram, or from 1 to 10 mmol/gram. The density refers to the concentration of ligand per gram (dry weight) of matrix.

The AAV vector ligand may be attached to the stationary phase by means described further below. For example, the AAV vector ligands may be attached to the matrix by amine binding. Alternatively, the AAV vector ligands may be attached by thiol binding.

The AAV vector ligand may have a binding affinity for one or more of AAV vector serotypes AAV1-AAV13, such as at least one of AAV1, AAV2, AAV3, AAV5, AAV6 and AAV10, and engineered variants of any of these.

According to a second aspect there is provided an affinity chromatography device comprising the stationary phase described above.

According to a third aspect there is provided a process for recovering adeno-associated virus (AAV) vectors from a solution, comprising: providing a solution comprising the AAV vector and one or more impurities, contacting the stationary phase described above with the solution (optionally by adding the solution to the stationary phase), and eluting the AAV vector from the stationary phase by contacting the stationary phase with an elution buffer.

By “impurities” is here meant that the solution in addition to the AAV vector may contain cell host debris, protein, genomic DNA, serum protein, some elements of medium, helper DNA, helper virus etc. Such a solution may be the harvest from culturing of (a) cell line(s) modified to produce the AAV vector.

As appreciated by persons skilled in the art, since the purpose is to bind the AAV vectors to the AAV vector ligands, the step of contacting the stationary phase with the solution is performed under conditions allowing said binding.

The solution may be added to the stationary phase directly from harvest or there may be an optional filtration step of the harvest before adding the solution to the stationary phase. The eluted solution comprises the AAV vector which is collected.

Advantageously, the time of contact between the stationary phase and the solution comprising the AAV vector during loading, also referred to as the residence time, can be very short. For example, the stationary phase may be contacted with the solution for a time period of less than 2 minutes, such as from 1 second up to 120 seconds, or from 1 second up to 60 seconds.

The amount of solution, such as clarified sample volume, added to the stationary phase may be up to 13 litres per mL of adsorbent volume of the stationary phase, which may be a packed stationary phase. Packed stationary phase here meaning that that the stationary phase is under some level of compaction. The adsorbent volume is the volume of the porous base matrix.

It is noted that the invention relates to all possible combinations of features recited in the claims.

### BRIEF DESCRIPTION OF THE DRAWINGS

These and other aspects of the present invention will now be described in more detail, with reference to the appended drawings showing embodiment(s) of the invention, in which:

FIG. 1 shows a non-woven polymer matrix comprising nanofibers.

FIG. 2 shows an affinity chromatography device comprising a stationary phase comprising the polymer matrix of FIG. 1.

FIG. 3. schematically illustrates a process of recovering adeno-associated virus (AAV) vectors from a solution.

FIG. 4a shows an exemplary reaction scheme of preparing the matrix material for immobilization of an AAV vector ligand. FIG. 4b shows an example of the linker chemistry that can be used to immobilise an AAV vector ligand onto the surface of cellulose acetate fibres.

FIG. 5 shows a graph of dynamic binding capacities onto the chromatography device of FIG. 2 across a range of residence times for two different AAV vectors.

As illustrated in the figures, some features maybe exaggerated for illustrative purposes and, thus, are provided to illustrate the general structures of embodiments of the present invention. Like reference numerals refer to like elements throughout.

### DETAILED DESCRIPTION

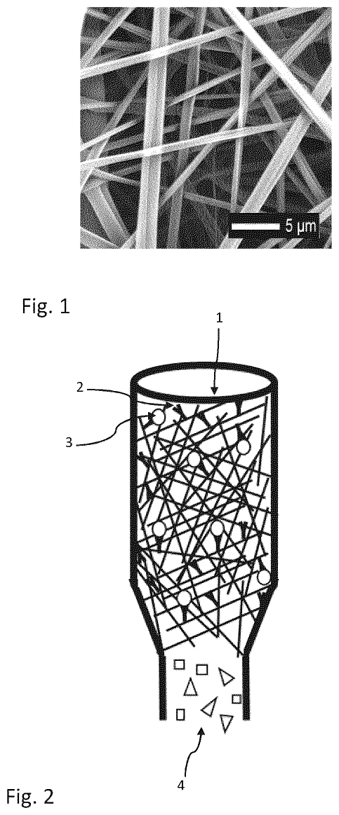

Below is described a stationary phase for use in affinity chromatography for recovering AAV vectors from a solution. The stationary phase comprises a non-woven polymer matrix 1 comprising nanofibers, see FIG. 1, wherein a mean flow pore size is 0.1-2.0 μm. Such a pore size is useful to enable virus particles (~20-200 nm in diameter for commonly used viral vectors) to enter the matrix 1.

The non-woven polymer matrix 1 comprising nanofibres is a mat of one or more polymer nanofibres with each fibre oriented essentially randomly, i.e. it has not been fabricated so that the fibre or fibres adopts a particular pattern. The non-woven polymer matrix 1 is typically provided by known methods. The non-woven matrix 1 may, in certain circumstances, consist of a single polymer nanofibre. Alternatively, the non-woven matrix 1 may comprise two or more polymer nanofibers.

The polymer nanofibres may be electrospun polymer nanofibres. Such electrospun polymer nanofibres are well known to the person skilled in the art. Alternative methods for producing polymer nanofibres may also be used, e.g. drawing.

The polymer nanofibres typically have mean diameters from 10 nm to 1000 nm. For some applications, polymer nanofibres having mean diameters from 200 nm to 800 nm or 200 nm to 400 nm may be appropriate.

The length of polymer nanofibres is not particularly limited. Thus, conventional processes e.g. electrospinning can produce polymer nanofibres many hundreds of metres or even kilometres in length. Typically, though, the one or more polymer nanofibres have a length up to 10 km, preferably from 10 m to 10 km.

The non-woven matrix 1 typically has a surface area from 1 to 40 g/m², from 5 to 25 g/m², from 1 to 20 or 5 to 15 g/m².

The non-woven matrix 1 typically has a thickness from 5 to 120 μm.

Suitable polymers include polyamides such as nylon, polyacrylic acid, polymethacrylic acid, polyacrylonitrile, polystyrene, polysulfones e.g. polyethersulfone (PES), polycaprolactone, collagen, chitosan, polyethylene oxide, agarose, agarose acetate, cellulose, cellulose acetate, dextran, and combinations thereof. Polyethersulfone (PES), cellulose and cellulose acetate are preferred. In some cases, cellulose and cellulose acetate are preferred. Cellulose acetate is readily formed into nanofibres, e.g. by electrospinning and can readily be transformed into cellulose after electrospinning. In some embodiments, the matrix comprises one or more nanofibres formed from different polymers. Typical polymers are as defined above.

The polymer matrix 1 is functionalised through attachment of AAV vector ligands 2 to the matrix 1, the AAV vector ligand 2 having a binding affinity for the AAV vector 3, see FIG. 2. A density of ligands 2 in the polymer matrix 1 is 0.1 μmol/gram-10 mmol/gram.

This functionalisation renders the matrix 1 comprising the AAV vector ligand 2 suitable as a stationary phase for use in affinity chromatography, see FIG. 2, for recovering AAV vectors 3 from a solution.

Prior to functionalising with the ligand, the nanofibres may optionally be physically modified, fused together at points where nanofibers intersect one another, by thermal or chemical methods and/or by pressing the polymer non-woven matrix. This may improve the structural stability of the matrix. The pressing and heating conditions may also be varied to alter the thickness and/or porosity of the resultant matrix.

Use of multiple non-woven matrices/sheets enables a thicker material to be prepared, which may have a greater capacity for adsorbance. The functionalised polymer matrix is typically therefore formed by providing two or more non-woven matrices stacked one on top of the other, each matrix comprising one or more polymer nanofibres, and simultaneously heating and pressing the stack of matrices to fuse points of contact between the nanofibres of adjacent matrices/sheets.

In the case of a cellulose matrix, this is typically formed by providing two or more non-woven matrices stacked one on top of the other, each said matrix comprising one or more cellulose acetate nanofibres, and simultaneously heating and pressing the stack of sheets to fuse points of contact between the nanofibres of adjacent matrices/sheets. The polymer matrix may consist of cellulose only. Alternatively, the matrix may comprise cellulose in combination with one or more polymer nanofibers.

Preferred processing conditions for pressing and heating of polymer nanofibres/non-woven sheets can e.g. be found in WO-A-2015/052460 and WO-A-2015/052465.

The nanofibres comprise one or more functional groups. Different functional groups may be present on different polymer nanofibres. Typical functional groups include hydroxyl, amino and carboxyl groups. Typically, the nanofibres are treated to introduce the one or more functional groups, or the nanofibres are treated to deprotect or activate any functional groups, or the nanofibres are treated to increase the number/density of functional groups.

For instance, when the matrix comprises cellulose, typically cellulose acetate nanofibres are provided and, prior to attaching the AAV vector ligand thereto, the cellulose acetate is treated to convert it to cellulose. This involves the deprotection of acetylated hydroxyl groups to give hydroxyl groups. Conversion of cellulose acetate to cellulose is typically effected using aqueous alkali, preferably NaOH in water: ethanol.

Derivatised cellulose, i.e. cellulose acetate, may be used to enhance solubility and/or other properties of the polymer to improve its suitability to be electrospun. Methods for increasing the number/density of functional groups on the substrate will be known to the skilled person.

The polymers used to form the nanofibres may be functionalised prior to the step of forming the nanofibres. Alternatively, and preferably, the nanofibres are functionalised after the polymer has been formed into nanofibres.

The ligands may typically be introduced by contacting the one or more nanofibres, which have been optionally pressed and/or heated and which optionally have one or more polymer chains covalently bonded thereto, with a reagent, which functionalises the product as a chromatography medium.

In general terms, the functionalisation of the medium/nanofibres changes their chemical and/or physical properties. This in turn affects how the functionalised chromatography medium behaves when used in a chromatography method. The modifications may, for example, change the polarity, hydrophobicity or biological binding properties of the functionalised chromatography medium compared to its unfunctionalised form. The modifications may, in certain circumstances, change more than one of the polarity, hydrophobicity or biological binding properties of the functionalised chromatography medium compared to its unfunctionalised form. In one embodiment, the modification changes the polarity and hydrophobicity of the functionalised chromatography medium compared to its unfunctionalised form.

Typically, the chromatography medium is functionalised with diethylethanolamine (DEAE), quaternary amine (Q), sulphopropyl (SP), carboxymethyl (CM), phenyl, or mercapto ethyl pyridine (MEP) groups. These ligands may be bonded to the polymer nanofibres and/or, where polymer chains have been covalently bonded to the nanofibres, may be bonded to the polymer chains.

As mentioned above, ligands may be attached to the nanofibres/polymer chains by treating with a suitably chosen reagent. 2-chloro-N,N-diethylamine hydrochloride (DEACH), glycidyltrimethylammonium, 1,4-butanesulfone, sodium chloroacetate, TEMPO followed by sodium perchlorate, or allyl gycidyl ether followed by sodium disulphite, styrene oxide, are examples of reagents which may be used.

Ligand groups are typically introduced into the functionalised chromatography medium by reacting a suitable reagent with one or more functional groups contained on the polymer nanofibres and/or polymer chains. Typical functional groups include hydroxyl, amino, halogen and carboxyl groups.

The one or more functional groups may be activated prior to reaction with a reagent. Conventional activation methods known in the art may be employed. Thus, in the case where the functional group is an hydroxyl group, such a group may be activated by treating with carbonyl diimidazole (CDI), bisoxiranes, cyanuric acid, N-hydroxysuccinimide esters (NHS),2-fluoro-I-methyl pyridinium toluene-4 sulphonate (FMP), NaI04, or divinylsulfone. In the case where the functional group is an amino group, such a group may be activated by treating with epichlorohydrine, glutaraldehyde or epoxide. In the case where the functional group is a carboxyl group, such a group may be activated by treating with CDI or I-ethyl-3-(3-dimethylaminopropyl) carbodiimide (EDC). In the case where the functional group is a halogen atom, such a group may be activated by treating with divinylsulfone.

A skilled person can choose suitable reagents to introduce particular groups and moieties onto particular nanofibres/polymer chains, for example on the basis of the desired ligand groups and moieties and the functional groups contained in those nanofibres/polymer chains.

AAV vector ligands 2 may be attached to the matrix by amine binding. When the functional group is an amino group, such a group may be activated be treating with e.g. epichlorohydrine, glutaraldehyde or epoxide. The matrix may be functionalized before or after the polymer(s) has been formed into nanofibers.

The AAV vector ligand 2 may be a camelid antibody or antibody fragment known in the art. Such camelid antibody/antibody fragment is an AAV ligand with affinity for binding AVV vectors. Such an AAV vector ligand has a binding affinity for one or more of AAV vector serotypes, for example AAV1, AAV2, AAV3, AAV5, and to a lesser extent AAV6, AAV8, and AAVrh10.

A total of 11 naturally occurring AAV serotypes have been isolated from animal tissues, AAV1-11. The different serotypes vary in their tissue tropisms and many other synthetic hybrid, modified or chimeric serotypes have also been engineered with the aim of enhancing or merging desirable traits for therapeutic benefit. The most commonly used serotypes depend somewhat on clinical targets and so are subject to change over time, but are currently rAAV2 and rAAV5. Such recombinant AAV vectors, rAAV, are used in all clinical trials and differ from wildtype serotypes in that two wildtype viral genes (rep and cap) have been removed, rendering the virus replication incompetent. rAAV also have a transgene expression cassette inserted between the two inverted terminal repeats (ITRs) to allow expression of the therapeutic gene(s) of interest.

When the stationary phase comprising the functionalised matrix 1 is used in affinity capture chromatography, a mobile phase, the solution, containing the AVV vector 3 is passed over the stationary phase 1 comprising the functionalised matrix and the AVV vector 3 is retained in the matrix 1 by the AAV vector ligand 2 in preference to other components 4 also present in the solution. Such other components 4 in the mobile phase may comprise impurities such as cell host debris, protein, genomic DNA, serum protein, some elements of medium, helper DNA, or helper virus, etc.

The stationary phase may be used in a chromatography system or used manually with syringes. All devices could be used with peristaltic pump, diaphragm pump, or positive gas pressure.

The density of ligand in the matrix may be determined by a titration method to determine the number of ligand moieties in the functionalised material. A skilled person will be aware of suitable methods to use to determine the amount of particular moieties present in a given sample of functionalised material. The functionalised matrix typically has a dynamic binding capacity (DBC), of the target entity, of 10 to 210 mg/ml (10% breakthrough), preferably 20 to 195 mg/ml (10% breakthrough), 30 to 180 mg/ml (10% breakthrough), 40 to 165 mg/ml (10%) breakthrough), or 50 to 150 mg/ml (10% breakthrough). In certain embodiments, the DBC may be up to 50 mg/ml (10% breakthrough), for instance 10 to 50 mg/ml (10% breakthrough). The DBC for 10% breakthrough can be determined in accordance with standard means, e.g. using an ÄKTA Pure liquid chromatography system.

The above described stationary phase has a high surface area for high binding capacity and a macroporosity needed for viruses to enter the matrix. The diameter of different viruses ranging from 20-300 nm. An AAV vector typically has a diameter of about 25 nm.

Due to the mean flow pore size of 0.1-2.0 μm and a density of ligands 2 in the polymer matrix 1 of 0.1 μmol/gram-10 mmol/gram when adding the mobile phase, the solution comprising the AAV vector 3, to the stationary phase the rate of binding of the AAV vector to the AAV vector ligand is merely dependent on the binding kinetics. The matrix 1 of the present stationary phase has an open pore structure where mass transfer is governed by convective flow. Hence, the use of the present stationary phase in affinity chromatography can be used with shorter residence times than when using traditional resin-based stationary phases.

Exemplary residence times applicable to the present invention may be less than 2 minutes, such as from 1 second up to 120 seconds, or from 1 second up to 60 seconds, such as from 1 to 10 seconds, from 1 to 5 seconds, from 1 to 45 seconds, or from 30 to 45 seconds.

The residence time may be adapted to the particular AAV serotype and the associated binding affinity of the AAV vector ligand. It is believed that at residence times relevant to the present invention (such as residence time of about 1 minute or less than 1 minute), the binding kinetics between the AAV serotype and the AAV vector ligand in question may be considered for the purpose of process efficiency. In contrast, the binding kinetics is not a typical consideration for affinity interactions on diffusive chromatography media, such as conventional resins. With resins, the rate limiting step is the time required for diffusion through the resin pores. Convective flow chromatography, as used in the present invention, has the potential to remove that bottleneck, enabling drastically shorter residence times, such that the binding kinetics may be a limiting factor for how short the residence times can be.

For example, in a process according to embodiments of the invention for recovering an AAV5 vector, the residence time may be less than 30 seconds, or less than 10 seconds, such as 1-5 seconds. In other embodiments, a process for recovering AAV2 vector may use a residence time of up to 60 seconds, such as 30-60 seconds, or about 30 seconds.

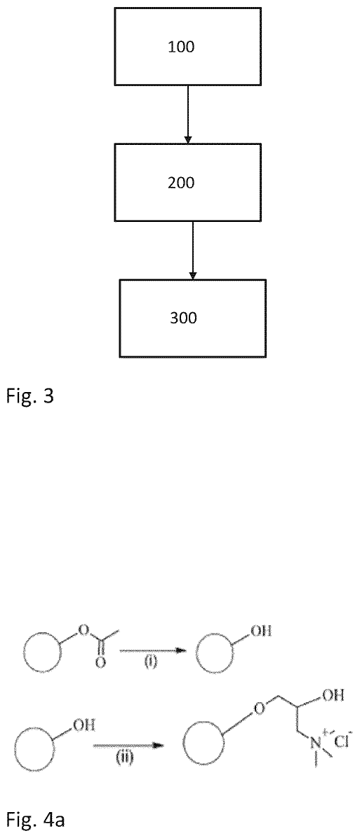

In FIG. 3 is schematically illustrated a process for recovering AAV vectors 3 from a solution. The process comprises steps of obtaining 100 a solution comprising the AAV vector 3 and one or more impurities 4. Adding 200 the solution to the stationary phase described above, and eluting 300 the AAV vector 3 from the stationary phase by contacting the stationary phase with an elution buffer.

The solution may be added to the stationary phase directly from harvest. Alternatively, there may be an optional filtration step (e.g. tangential flow filtration (TFF)) of the harvest before adding the solution to the stationary phase.

Between the step of adding 200 the solution to the stationary phase and the eluting step 300, the process may comprise a step of washing the stationary phase to which is adsorbed the viral product and/or product-related and/or non-product-related impurities. This washing step is carried out to remove any components which are not bound to the AVV vector ligand 2. This can be carried out in accordance with conventional methods known for the washing phase of such processes. This washing step typically involves washing with a liquid phase of low ionic concentration.

The eluted solution comprises the AAV vector 3 being collected. Typically, the process of recovering the AAV vector 3 comprises a single bind-elute step or a single flow-through step. Alternatively, the process may comprise more than one bind-elute step in series, e.g. two, three, four, five or more bind-elute steps. Alternatively, the process may comprise more than one flow-through step in series, e.g. two, three, four, five or more flow-through steps. Alternatively, the process may comprise a combination of bind-elute and flow-through steps in series, e.g. two, three, four, five or more steps in total.

After the elute step 300, the process may further comprise a step of regenerating the matrix. Typically this is effected by contacting the matrix from which the viral product and/or product related impurities have been eluted with buffer. This can be carried out in accordance with conventional methods known for the regeneration phase of such processes.

Typically, the product fraction contains a greater amount of AAV vector expressed as a percentage of the total amount of viral product and product-related impurities than was present in the solution. Typically, the amount of AAV vector in the product fraction expressed as a percentage of the total amount of viral product and product-related impurities is greater than the amount in the solution by a factor of 10 or more times.

In the process illustrated in FIG. 3, the flow rate used is dependent on the dimensions of the stationary phase and the residence time chosen. Feasible residence times in the process would be 0.1 s-2 min. Equivalent flow velocity in a 0.4 ml lab scale unit would be about 3000-2 cm/h. In a 2.4 litre unit a maximum feasible flow velocity over the stationary phase would be about 850 cm/h.

The amount of input solution added to the stationary phase in the process may be up to 13 litres per ml adsorbent volume of the packed stationary phase, without clogging and fowling of the matrix. Thereby, the present stationary phase and chromatography device using such stationary phase provides sufficient volumetric loading capacity for typical AAV bioprocess feeds to reach the dynamic binding capacity of the packed stationary phase.

While the invention is described herein with respect to exemplary embodiments, the skilled person will appreciate that the invention is not limited to these. In the claims, any reference signs placed between parentheses shall not be construed as limiting the claim. Use of the verb “to comprise” and its conjugations does not exclude the presence of elements or steps other than those stated. The article “a” or “an” preceding an element does not exclude the presence of a plurality of such elements. The mere fact that certain measures are recited in mutually different dependent claims does not indicate that a combination of these measures cannot be used to advantage.

### EXPERIMENTAL

### Example 1: Pore Size Measurement Method

The mean flow pore size can be measured using capillary flow analysis using commercially available equipment. In an example, the equipment used was a POROLUX™ 100 porometer (IB-FT GmbH, Berlin, Germany) according to the manufacturer's manual and methodology was as given in Table 1.

TABLE 1
Capillary flow porometry

| Porometer | Porometer model | Porolux 100 | |
|---|---|---|---|
| specifications | Porometer algorithm | Pressure Scan | |
| | Measurable pore size | 90 nm-500 μm | |
| | Pressure range | 0-7 | bar |
| | Flow rate | up to 100 liters per minute | |
| | First Bubble Point | Calculated - according to | |
| | | ASTM F 316-03 | |
| Settings | Calculated bubble | First Flow | |
| | point method used | | |
| | Slope of pressure increase | 6.25 mbar/s (160 s/bar) | |
| | Number of data points | 50 | |
| | for wet curve | | |
| | Number of data points | 25 | |
| | for dry curve | | |
| | Test Gas used | Nitrogen | |
| | Fluid angle (°) | 0 (Default) | |
| | Shape factor name | 1 (Default) | |
| | Shape factor | 1 (Default) | |
| | Temperature | 21 | °C |
| Wetting fluid | Wetting Fluid | Porefil (Perfluoropolyether) | |
| | Surface Tension | 16.45 ± 0.02 | mN/m |
| | Tensiometer method | 22 °C, 40% RH | |
| | for Wetting Fluid | | |
| | surface tension | | |
| | Viscosity | 2.2 | mPas |

### Example 2: Production of Stationary Phase for Use in Chromatography

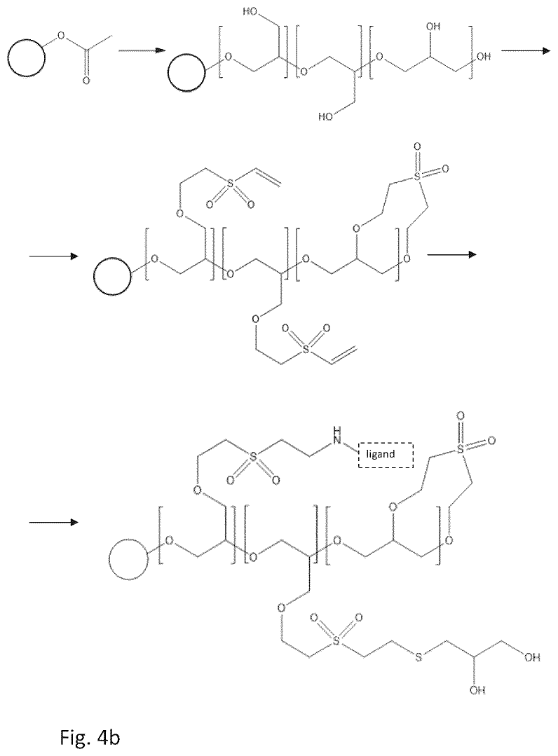

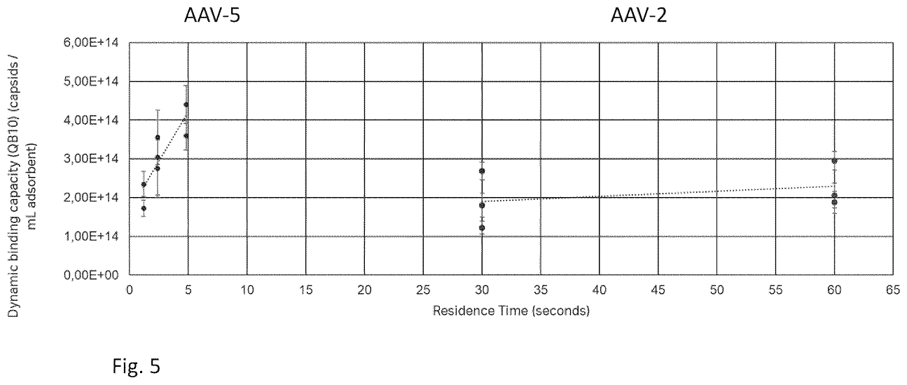

Below follows a non-limiting example of production of a polymer matrix 1, immobilization of a ligand 2 therein and the use of the thus formed stationary matrix in an affinity chromatograph. FIG. 4a shows a reaction scheme of preparing the matrix 1 material for immobilisation of the AAV vector ligand 2. FIG. 4b shows the linker chemistry used to immobilise the AAV vector ligand 2 onto the surface of cellulose acetate fibres.

### Sheet Production

The matrix material may be produced as described in WO2018/011599, or as to produce a laminated non-woven sheet of fibres. A solution of cellulose acetate (CA) with a relative molecular mass of 29,000 g/mol is dissolved in a binary mixture of glacial acetic acid and ethanol in a 3:1 ratio. This is the primary solution. When they are mixed, polyethethylene oxide dissolved in deionised (DI) water to a concentration of 5% is then added to the primary CA solution in a quantity of 1.2% of the total volume of CA prior to electro spinning to produce fibres with diameters ranging between 300-600 nm. Optimised conditions for nanofibre production can be found in, for example, O. Hardick, et al, J. Mater. Sci. 46 (2011) 3890. Sheets of approximately 20 g/m² material were layered and subjected to a combined heating and pressure treatment.

The matrix material thus formed has a mean flow pore size of 0.1-2.0 μm, which may be measured using bubble point porometry (Porolux, Porometer NV). Through varying a combination of standard electrospinning parameters and reducing the number of fibre layers, mean flow pore sizes across this range can be reliably obtained.

### CA Pre-Washing

35× sheets of the CA material (100×155 mm2) are sandwiched between gauze and loaded into a flow reactor. The material is washed by recirculation of 5 L DI water for 20 min. The reactor is emptied and the washing process is repeated a further 2 times, with material being stored overnight in the final wash if necessary.

### Glycidol Step

In an 8 L beaker, KOH (265 g) was added to DI water (4723 mL). The solution was stirred vigorously. Upon complete dissolution, glycidol (1350 mL or 675 mL, dependent on 100% or 50% glycidol base matrix, respectively) was added and stirred vigorously for 4 min. The reactor was emptied from the washing water and the KOH/glycidol solution was added to the reactor. The recirculating pump was started. A reaction temperature profile of 20° C. was used and the solutions continuously flown through the membranes to prevent the reaction temperature exceeding 20° C. After 6-7 h of recirculation, the reacting solution was removed, and the material was washed by recirculation of 5 L of DI water for 20 min and then emptied. The washing process was repeated at least 3 times (or as many times as necessary until the final pH is neutral). The sheets were stored in DI water overnight.

### Saponification Step

Material made and referred to as 0% glycidol is known as regenerated cellulose (RC) and is synthesised in a saponification reaction where the terminal acetate groups on the cellulose acetate backbone are cleaved, to leave alcohol groups. This step occurs in substitution of the glycidol step, and is followed by the divinyl sulfone (DVS) step.

In an 8 L beaker, KOH (132 g) was added to DI water (3149 mL), with EtOH (1574 mL). The solution was stirred vigorously until complete dissolution. The washing water is emptied from the flow reactor and KOH/EtOH solution added. The recirculating pump is started and run for 6 h at 22-24° C. After this time, the reaction mixture was removed and 5 L DI water is added to the reactor. The recirculating pump is started and run for 20 min. The washing water is removed. The water wash is repeated a further 3 times.

### Divinyl Sulfone (DVS) Step

The flow reactor was emptied from the washing water. In an 8 L beaker, Na2CO3 (316 g) was added to DI water (4211 mL). The solution was vigorously stirred until complete dissolution. Acetonitrile (1258 mL) was added under vigorous stirring. The solution was added to the flow reactor. The recirculating pump was started for 4 min, before DVS (1350 mL) was added carefully in one portion to the reaction vessel.

After 6 h of recirculation, the reaction mixture was removed. A 1:1 acetone/DI water (5 L) was added to the flow reactor and the recirculating pump was started and ran for 20 min. The washing solution was removed. The acetone/water washing process was repeated a further 3 times.

DI water (5 L) at 22-24° C. was added to the flow reactor and the recirculating pump was started and ran for 20 min. The washing water was removed. The water washing process was repeated once.

### Immobilization

The AAV vector ligand of choice can be immobilized in the matrix. In this example, a spin filtered solution of AAV binding ligand having a concentration of 2.5 mg/ml was used for coupling.

A coupling solution of 3.0 M (NH4)2SO4, 0.1 M NaHCO₃ was prepared and adjusted to pH 9.

A sheet of DVS treated matrix material was placed into a sealable container (155×105 mm2) and an amount of ligand solution at the desired concentration added with an amount of coupling solution to make up the desired total volume, as shown in Table 2. The container was sealed and placed on an orbital shaker for 16 h at 22-24° C. After this time, the supernatant was collected. All sheets were washed with DI water for 20 min. This was repeated a further 3×, collecting the wash supernatant each time for later quantification of the immobilisation efficiency.

### Blocking

Either blocking with ethanolamine or blocking with thioglycerol was carried out.

Blocking with ethanolamine: A blocking solution of 0.3 M ethanolamine was adjusted to pH 9 and 25 mL dispensed onto each sheet. The containers were sealed and placed on an orbital shaker for 16 h at 22-24° C. After this time, the blocking solution is discarded and sheets were washed with DI water for 20 min. This was repeated once. Sheets were washed with PBS adjusted to pH 2.0 for 20 min, followed by PBS at pH 7.4. This two-step process was repeated once, followed by 2×DI water washes for 20 min each.

Blocking with thioglycerol: A blocking solution of 0.288M thioglycerol, 0.1 M Na2HPO4·12H2O, 0.001 M EDTA was adjusted to pH 8.3 and 25 mL was dispensed onto each sheet. The containers were sealed and placed on an orbital shaker for 16 h at 22-24° C. After this time, the blocking solution was discarded and sheets washed with DI water for 20 min. This was repeated once. Sheets were washed with 0.5 M AcOH for 20 min, followed by 0.1 M Tris, 0.15 M NaCl at pH 8.5. This two-step process was repeated once, followed by 2×DI water washes for 20 min each.

After blocking has been performed the material is fully immersed in 1:1:3 glycerol/ethanol/water and stored in the fridge for a minimum of 1 hour.

### Ligand Density Measurement

Using a NanoDrop spectrophotometer, the AAV vector ligand concentration of each collected supernatant was calculated. This was used to calculate the mass of ligand immobilised. One disc was taken from each sheet where the supernatant was collected/concentration measured as described above. The thickness of each disc was taken at five points across the sheet, using a Mitutoyo Micrometer, to calculate an average thickness for the sheet. From this, the total volume of the sheet was calculated. The mass of ligand immobilised was divided by the volume of the sheet to calculate the ligand density. An average ligand density can be calculated for each batch.

TABLE 2
Details values of unspecified parameters in experimental procedure.

| | | | | Conc. | | |
|---|---|---|---|---|---|---|
| Amount | Volume | | | of | Volume | |
| of | of | Time of | Total | ligand | coupling | |
| glycidol | glycidol | glycidol | volume | (mg/ | solution | Blocking |
| (%) | (mL) | rxn (h) | (mL) | mL) | (mL) | soln. |
| 0 or 50 | 675 or | 6 or 7 | 25 or | 0.5-6.0 | 17 or 34 | Thioglyerol |
| or 100 | 1350 | | 50 | | | or |
| | | | | | | ethano- |
| | | | | | | lamine |

With the above described procedure a ligand density of 3-7 mg/ml adsorbent is obtained, which for some ligands may correspond to approximately 0.5-5 μmol/g, e.g. 0.7-4.6 μmol/g). With such a ligand density it is possible to achieve static binding capacities in excess of 1E15 AAV-5 capsids per mL of adsorbent, when AAV-5 is used. The ligand density being measured using amino acid analysis or UV spectroscopic analysis of the pre-, post- and wash immobilisation solutions.

### Example 3: Use of the Matrix with Immobilized AAV Vector Ligand

The AAV vector recovery from matrix material with immobilized AAV vector ligand represents the fraction of AAV obtained after purification with the present matrix material (considering that the process has associated losses of AAV) and it is defined only for a loading of 85% capacity at the residence time tested and for that particular AAV serotype. Smaller loads or shorter residence time may result in lower recovery. Purity is defined as the reduction in bioburden content, i.e., reduction in host cell DNA and protein.

### AAV Vector Production

The AAV vector material is typically generated from cells that have been modified to produce the constituent parts of the vector e.g. its coat or capsid, and the nucleic acid material that is intended to be delivered to the cells. After lysis and clarification, the “crude materials” contains vectors, cell host debris, protein, genomic DNA, serum protein, some elements of medium, helper DNA, helper virus etc., which constitutes the input feed for subsequent purification (see below). Below follows an example of a cell culturing method, transfection and harvest.

### Culturing

### Materials:

FreeStyle 293-F cells (Thermo Fisher Scientific)
Hycell TransFx-H (GE Healthcare) supplemented with 4 mM Glutamax (Thermo Fisher Scientific) and 0.1% (v/v) Pluronic F-68
DMSO (Dimethyl Sulfoxide)

### Thawing Cells:

Add 9 mL pre-warmed culture medium to a 15 ml tube.
Thaw quickly the cell vial in a 37° C. water bath.
When only a small portion of ice in present, move the vial to the BS cabinet. With the pipette, gently resuspend the content of the vial and pipette it slowly into the 15 ml tube.
Dilute in a final volume of 30 ml of culture medium in a T75 tissue culture flask and place in a cell incubator set to 37° C. and 8% CO₂.
After 3-4 days, measure the cell density. If a minimum of 2E7 viable cells are in culture dilute them in a 100 mL spinner flask. Set agitation to 130 rpm.

### Passage Cells:

Cell should be passaged at approximately 1-2E6 cells/mL (usually every 2-3 days).
Measure cell density and viability in the culture. Also visually assess aggregation in the cell suspension using a microscope. If there is a high degree of aggregation, strongly pipette the cell suspension (~20×) or vortex for a few seconds.
Dilute cell suspension to 0.2-0.5E6 cells/mL in fresh pre-warmed culture media and return to the cell incubator.

### Freezing Cells:

Measure cell density and viability in the culture (viability should be >95%).
Spun down cell suspension and dilute the cells to approximately >1E7 cells/ml in 50% conditionate medium and 50% fresh medium.
Add 925 μL of cell suspension to a cryovial.
Add 75 μL of DMSO.
Put the vials in a cell freezing container (surrounded by isopropyl alcohol) and keep it at −80° C. overnight.
Move to liquid N₂ cell bank for long-term storage.

### Transfection of Cells

### Materials:

FreeStyle 293-F cells
Hycell TransFx-H (Cytiva) supplemented with 4 mM Glutamax (Thermo Fisher Scientific)
Opti-MEM I reduced serum medium (Thermo Fisher Scientific)
Transporter 5 transfection reagent (Polysciences)
Supercoiled Rep/Cap plasmid “pRC” (1 mg/ml)
Supercoiled vector genome, typically a reporter or therapeutic gene flanked by AAV-2 inverted terminal repeat sequences “pGFP” (1 mg/mL)

Evening before transfection: Replace and/or add a minimum of 60% fresh medium (no Pluronic) to the cell suspension. Final cell density should be ~0.7-0.8E6 cells/mL. For transfection (1 L scale), next day:
Add 500 μL of pRC and 500 μl of pGFP plasmids (1 mg/ml each) to 47 mL of Opti-MEM at RT. Vortex briefly.
Add 2 mL of Transporter 5 transfection reagent to the DNA mix. Vortex 5 s. Incubate 20 min at RT.
Pipette up and down 3×.
Add to the cell suspension all at once.

### Harvest of AAV

### Materials:

Triton-X 100 (Sigma)
1 M MgCl₂ solution
Benzonase (Merck).
Pluronic F-68 solution (Thermo Fisher Scientific)
4 M NaCl solution

### Methods:

### 72 h Post-Transfection:

Add directly to each cell bag (1 L scale) in 10 mL PBS:

| 0.1% Triton-X 100 | 1 | mL |
|---|---|---|
| 2 mM MgCl₂ | 2 mL (1M | |
| | solution) | |
| 50 U/mL Benzonase | 200 | μL |
| 0.001% pluronic | 100 | μL |

Incubate for 3 h at 37° C.
Add 115 mL of 4 M NaCl solution (to increase NaCl concentration to 500 mM) and incubate 30 min.
Filter through ULTA Prime GF 5.0 μm and ULTA Prime CG 0.2 μm filter in series.
Take a sample for titration and store at −80° C.
Clarify and concentrate using a 300 kDa Pellicon TFF (tangential flow filtration) membrane cassette (Merck).

### Dynamic Binding Capacity

Below is described a dynamic binding capacity (QB10) test. Typically such a capacity test is performed first, then a separate run loaded to 85% of the dynamic binding capacity to evaluate purity/recovery (described further down). The data in FIG. 5 shows the results of dynamic binding capacity tests performed at a range of residence times for different serotypes.

### Equipment

AKTA Avant 150 (Cytiva)
CLARIOstar Plus microplate reader (BMG Labtech).
HiTrap chromatography unit (Cytiva) in which the matrix with immobilised AAV vector ligand was mounted

### Chemicals

Running buffer: 20 mM Tris, 500 mM NaCl, 0.001% Pluronic F-68 (Sigma Aldrich), pH 8.5
Elution buffer: 100 mM NaOAc, 500 mM NaCl, 0.001% Pluronic F-68 (Sigma Aldrich), pH 2.5
Neutralization buffer: 200 mM Tris, 500 mM NaCl, 0.001% Pluronic F-68 ((Sigma Aldrich)), pH 10.5
Input feed: approximately 2E14 capsids of post-TFF (tangential flow filtration), post charged depth-filtered AAV feed, at a concentration of 1E12 capsids/ml, in running buffer and filtered through a 0.2 μm filter immediately before use
AAV Titration ELISA kit of the appropriate serotype for the input feed (e.g. Progen Biotechnik GmbH, article number PRAAV5)

### Purification, AKTA Run.

Fill A1 and sample lines with running buffer and B1 line with elution buffer (or equivalent set up) and flush all the system with running buffer.
Dilute the input feed to ~1.3E12 capsids/mL in running buffer and filter it through a 0.2 μm.
Prime the sample line with feed, the remaining feed supply should be equivalent to roughly 2E14 capsids of diluted input feed (~1.3E12 capsids/mL).
Connect HiTrap unit to column valve.
Fill the sample collection tray with 50 mL and 15 mL tubes. Add 3 mL of neutralization buffer to the second 50 ml tube in the collection tray.
Run AKTA method as detailed below:
   Note: Parameters not detailed here are to be left as default.
   Method Settings:
      Pre-column pressure limit: 1.4 MPa
      Delta column pressure: 1 MPa
      Flow-rate: 10 mL/min
      Select column position where the HiTrap unit was connected to the AKTA
      UV1: 280 nm; UV2: 254 nm
   Equilibration 1:
      Volume: 10 mL
      Inlet B1: 100
   Equilibration 2:
      Volume: 10 mL
      Inlet B1: 0%
   Sample Application:
      Use the same flow rate as in Method Settings: tick
      Inject sample directly onto column: tick
      Sample inlet: S1
      Inject all sample using air sensor: tick
      Use the same inlets as in method settings: tick
      Inlet B: 0%
      Fractionate: using faction collector
      Fraction type: Fixed volume fraction
      Fraction destination: 15 ml tube
      Fixed fractionation volume: 10 mL
   Colum Wash:
      Inlet B: 0%
      Wash until the total volume: 20 mL
      Fractionate: using faction collector
      Fraction type: Fixed volume fraction
      Fraction destination: 50 mL tubes
      Fixed fractionation volume: 50 ml
   Elution:
      Isocratic elution volume: 20 mL
      100% of B
      Fill the system with selected buffer: tick
      Fractionate: using faction collector
      Fraction type: Fixed volume fraction
      Fraction destination: 50 mL tubes
      Fixed fractionation volume: 50 mL
   Equilibration 3:
      Volume: 4 mL
      Inlet B1: 0%
Collect 1 ml sample of each fraction (input, FT fractions, post-load wash and elution) and store in a mapped deep-well 96-well block, cover with adhesive lid.
If analysis is not performed within 24 h, store block at −80° C., otherwise store at 4° C.

### Dynamic Binding Capacity Estimation (QB10):

Perform an AAV titration ELISA on the samples collected, according with the manufacturer's recommended method. Each sample should be quantified in triplicate using 3 independent dilutions. A minimum of a 3-fold dilution is recommended to prevent potential matrix effects.
Calculate the volume (post injection) at which the conductivity is equal to 50% of the difference between the running buffer and the input feed. This is the dead volume between the sample line and the conductivity monitor and should be subtracted from the volume of input feed recorded by the AKTA to obtain a more accurate input volume.
Use a 5-parameter curve fit generated with the ELISA standards to determine the total amount of AAV particles in each fraction.
   Note: The total AAV capsids in the input feed is calculated using the adjusted input volume (input volume minus system dead volume).
Calculate the breakthrough (% BT) for each FT fraction i using the following expression and plot a BT curve. Fit a linear (or exponential) curve to the data points.

$$\% BT = \frac{AAV concentration in FT fraction i}{AAV concentration in input feed} \times 100$$

Calculate the total number of capsids for BT of 10% (HiTrap capacity).
Calculate the QB10 to 1 mL membrane, considering that HiTrap volume is 0.4 mL.

$$QB 10 = \frac{HiTrap capacity}{HiTrap volume}$$

### Purity/Recovery

After the dynamic test has been performed, a capacity test as described below may be performed wherein 85% of the dynamic binding capacity is loaded to evaluate purity/recovery.

### Equipment

AKTA Avant (Cytiva)—chromatography system.
CLARIOstar Plus microplate reader (BMG Labtech).
HiTrap chromatography unit (Cytiva) in which the matrix with immobilized AAV vector ligand is mounted.

### Chemicals

Running buffer: 20 mM Tris, 500 mM NaCl, 0.001% Pluronic F-68 (Sigma Aldrich), pH 8.5
Elution buffer: 100 mM NaOAc, 500 mM NaCl, 0.001% Pluronic F-68 (Sigma Aldrich), pH 2.5
Neutralization buffer: 200 mM Tris, 500 mM NaCl, 0.001% Pluronic F-68 (Sigma Aldrich), pH 8.5
Input feed: approximately 85% of calculated QB10 of post-TFF post-DF AAV feed, at a concentration of 1E12 capsids/ml, in running buffer and filtered through a 0.2 μm filter immediately before use
AAV Titration ELISA kit of the appropriate serotype for the input feed (e.g. Progen Biotechnik GmbH, article number PRAAV5)
Pierce Coomassie (Bradford) Protein Assay Kit (Thermo Fisher Scientific, article number 23200).
Quant-iT™ PicoGreen® dsDNA kit (Invitrogen, article number P7589). HEK 293 Host Cell Protein ELISA (Cygnus Technology, article number F650S). HCP ELISA sample diluent (Cygnus Technology, article number 1700)

### Purification, AKTA Run:

Fill A1, Sample and Out 1 lines with running buffer and B1 line with elution buffer (or equivalent set up) and flush all the system with running buffer.
Feed the estimated amount of input feed (85% of calculated QB10 at 1E12 capsids/mL) into the sample line.
Connect the HiTrap unit comprising matrix with immobilised ligand to the column valve.
Fill the sample collection tray with 96-deep well plates, 50 mL and 15 mL tubes.
Prepare and run a method to 1) load the desired volume of input feed onto the HiTrap unit at a residence time of 2.4 seconds, collecting the flow-through in fractions, 2) wash with 20 mL of running buffer, collecting the output as a single fraction, 3) elute with 20 ml of elution buffer, collecting as a single fraction.
Within 15 min of completing the run, add 3 mL of neutralization buffer to the elution fraction.
Collect 1 ml sample of flow-through, post-load wash fractions and input feed and store in the mapped deep well 96-well sample block where elution fractions were collected and covered with adhesive lid.
If analysis is not performed within 24 h, store at −80° C., otherwise store at 4° C.

### Evaluation, Quantification of Recovery:

Perform an AAV titration ELISA on the samples collected, according to the manufacturer's recommended method. Each sample should be quantified in triplicate using three independent dilutions. A minimum of 3-fold dilution is recommended to prevent potential matrix effects.
Calculate the volume (post injection) at which the conductivity is equal to 50% of the difference between the running buffer and the input feed. This is the dead volume between the sample line and the conductivity monitor and should be subtracted from the volume of input feed recorded by the AKTA to obtain an accurate input volume.
Use a 5-parameter curve fit generated with the ELISA standards to determine the total amount of AAV particles in each fraction.
Define the elution peak and elution peak fractions so it includes only the fractions whose AAV concentration is >10% of the most concentrated fraction.
Recovery can be determined using the following equation:

$$Recovery ( \% ) = \frac{\sum total AAV capsids in elution peak fractions}{total AAV capsids in the input feed} \times 100$$

The total AAV capsids in the input feed is calculated using the adjusted input volume (input volume minus system dead volume).

Quantification of purity:
1. Determine DNA amount in each sample using the Quant-iT™ PicoGreen® dsDNA Kit and according with the manufacturer's recommendation. Each sample should be quantified in triplicate using the high range calibration curve (linear fit). If elution samples are below the limit of detection, they should be measured using the low range calibration curve.

$$DNA reduction ( \% ) = ( 1 - \frac{\sum total DNA in elution peak fractions}{total DNA in the input feed} ) \times 100$$

2. Determine total protein concentration in each sample using the Pierce Coomassie (Bradford) Protein Assay Kit, according with the manufacturer's recommendation. Each sample should be quantified in triplicate using the high range calibration curve (5-parameters curve fit). If elution samples are below the limit of detection, they should be measured using the low range calibration curve. Sample and standard dilutions should be prepared in DNA, RNA-free water. If the protein concentration is below the limit of detection of the Pierce Coomassie assay, samples should be quantified using HEK 293 HCP ELISA, according with the manufacturer's recommendation. Three independent dilutions for each sample should be prepared in Cygnus sample diluent buffer.

$$Protein reduction ( \% ) = ( 1 - \frac{\sum total protein in elution peak fractions}{total protein in the input feed} ) \times 100$$

### Results

The dynamic binding capacities (at 10% breakthrough) of the matrix with immobilised AAV vector ligand were calculated for cell lysate feeds containing AAV serotypes AAV-2 and AAV-5, at residence times ranging from 1.2 seconds to 60 seconds. The results, shown in FIG. 5, demonstrate successful purification of AAV vectors at residence times ranging from a few seconds up to about 60 seconds. It was also found that the capacity for binding these AAV serotypes differed, presumably due to different binding kinetics between the AAV ligand with each serotype. It was also seen that altering the residence time had different levels of impact on capacity between these serotypes. Increasing AAV-5 residence time 4-fold led to a 2-fold increase in dynamic binding capacity. Whilst, for AAV-2, a plateau of dynamic binding capacity was achieved at 30 to 60 seconds of residence time, and shorter residence time will cause a reduction in capacity (data not shown). In essence, at residence times relevant for the present stationary phase, <60 seconds, the serotype of AAV may have a certain influence on the ligand-AAV binding kinetics. Notably, at residence times relevant for conventional resin chromatography, >60 seconds, serotype dependent ligand-AAV binding kinetics are not observed.

### Example 4: Large Scale Recovery

Recovery of AAV5 vector from a large-scale batch was tested. A 200 L batch of cell lysate feed was produced in a stirred tank bioreactor (Xcellerex XDR-200, Cytiva, Sweden) by scaling up the method described above, followed by clarification by depth filtration. Next, approximately 100 L of cell lysate feed was loaded on a 40 mL unit of the present stationary phase, prepared as described in Example 2 above and run at 10 seconds residence time, using buffers and conditions as outlined in Table 3.

TABLE 3
Large scale recovery

| | | | Column | Flow |
|---|---|---|---|---|
| Step | Buffer | | volumes | rate |
| Conditioning | 0.1M glycine, pH 2.5 | | 25 | 240 mL/min |
| Equilibration | 0.02M Tris, 0.5M NaCl, pH 8 | | 25 | |
| Load | AAV vector depth | Run 1 | 3852 | |
| | filtrate | Run 2 | 3748 | |

| | | |
|---|---|---|
| Wash 1 | 0.02M Tris, 0.5M NaCl, pH 8 | 35 |
| Wash 2 | 0.02M Tris, pH 7.5 | 35 |
| Elution | 0.1M glycine pH 2.5 | 20 |
| Strip | 0.1M phosphoric acid, 2.0M NaCl | 10 |

The resulting recovery of AAV5 was 90-100%.

1. A stationary phase for use in affinity chromatography for recovering adeno-associated virus (AAV) vectors from a solution comprising said AAV vectors, the stationary phase comprising a porous base matrix having a mean flow pore size of 0.1-2.0 μm, and AAV vector ligands attached to the matrix, the AAV vector ligands having a binding affinity for the AAV vector, wherein the stationary phase has a density of ligands in the matrix in the range of from 0.1 μmol/gram to 10 mmol/gram.

2. The stationary phase of claim 1, wherein the porous base matrix is a non-woven polymer matrix.

3. The stationary phase of claim 2, wherein the non-woven polymer matrix comprises nanofibers, wherein the nanofibres optionally comprise at least one hydrophilic polymer selected from: cellulose, polyethersulfone, polystyrene methyl acrylate, dextran and agarose.

4. The stationary phase according to claim 1, wherein the mean flow pore size is in the range of from 10 nm to 1 μm, such as from 200 nm to 1 μm.

5. The stationary phase according to claim 1, wherein the ligand density is in the range of from 0.1 to 10 μmol/g, such as 0.2 to 10 μmol/g, such as 0.5 to 5 μmol/g.

6. The stationary phase of claim 1, wherein the AAV vector ligands are attached to the matrix by amine binding or thiol binding.

7. The stationary phase of claim 1, wherein the AAV vector ligand is an antibody or antibody fragment.

8. The stationary phase of claim 1, wherein the AAV vector ligand has a binding affinity for one or more of AAV vector serotypes AAV1-AAV13, such as one or more of AAV1, AAV2, AAV3, AAV5, AAV6 and AAV10, and engineered variants thereof.

9. An affinity chromatography device comprising the stationary phase of claim 1.

10. Use of a stationary phase according to claim 1, for purifying an AAV vector from a solution comprising the AAV vector and one or more impurities, wherein the AAV vector optionally is selected from AAV1, AAV2, AAV3, AAV5, AAV6 and AAV10, and engineered variants thereof.

11. A process for recovering adeno-associated virus vectors from a solution, comprising:
providing a solution comprising the AAV vector and one or more impurities,
contacting a stationary phase according to claim 1, with said solution,
eluting the AAV vector from the stationary phase by contacting the stationary phase with an elution buffer.

12. The process of claim 11, wherein the amount of solution added to the stationary phase is up to 13 litres per ml adsorbent volume of a packed stationary phase.

13. The process of claim 11, wherein the step of contacting comprises adding the solution to the stationary phase.

14. The process of claim 11, wherein the solution forms a mobile phase, and the stationary phase is contacted with the solution during a residence time in the range of from 1 to 60 seconds, or less than 60 seconds.

15. The process of claim 11, wherein the AAV vectors are of AAV serotype 1, 2, 3, 5, 6 or 10, or an engineered variant thereof.
